(19) United States
(12) Patent Application Publication
(10) Pub. No.: US 2024/0000065 A1
(43) Pub. Date: Jan. 4, 2024

(54) METHOD AND SYSTEM FOR EX-SITU NORMOTHERMIC HEART PERFUSION

(71) Applicant: "National Research Cardiac Surgery Center" NPJSC, Nur-Sultan (KZ)

(72) Inventors: Yuriy Pya, Nur-Sultan (KZ); Rymbay KALIYEV, Nur-Sultan (KZ); Timur LESBEKOV, Nur-Sultan (KZ); Linar FAIZOV, Nur-Sultan (KZ); Zhuldyz NURMYKHAMETOVA, Nur-Sultan (KZ); Assel MEDRESSOVA, Nur-Sultan (KZ); Bauyrzhan ZHUNUSSOV, Nur-Sultan (KZ); Anatoliy SERGIYENKO, Nur-Sultan (KZ); Vladislav KAPINOSSOV, Nur-Sultan (KZ); Aidyn KUANYSHBEK, Nur-Sultan (KZ)

(21) Appl. No.: 18/055,125

(22) Filed: Nov. 14, 2022

(30) Foreign Application Priority Data
Jun. 30, 2022 (KZ) 2022/0415.1

(51) Int. Cl. A01N 1/02; A61M 60/232

(52) U.S. Cl. CPC A01N 1/0247; A61M 60/232

(57) ABSTRACT

The invention has medical application. More particularly, in various ernbodiments, the invention applies specifically to Cardiac surgery, Transplantology and Extracorporeal perfusion medicine. The invention aims to create a system for normothermic preservation and long-term transportation of a donor heart with additional properties universality, accessibility. The described system includes a special integrated box reservoir for donor heart placement, oxygenator, centrifugal pump and lines connecting all components. Normothermic, oxygenated blood with the necessary solutions is pumped under controlled pressure into the aorta for myocardium blood supply through the coronary arteries. Venous blood from the heart cavities enters the integrated box reservoir, and pressurized into the oxygenator by centrifugal pump. Then the oxygenated perthsate is pumped into the aorta, closing the circulatory loop of the system, The capacity of the device is monitored and controlled by an integrated electronic system. A special gas blender controls oxygen and gas components supply. The system lines provide integrated ports for hemocorrection and hemodiafiltration modules, and for laboratory sampling the perfusate,

### FIELD OF THE INVENTION

The invention generally relates to systems and methods for ex-situ heart perfusion, preservation and therapy. More particularly, in various enibodiments, the invention applies specifically to Cardiac surgery, Transplantology and Extracorporeal perfusion medicine.

### BACKGROUND OF THE INVENTION

The Organ Care System (OCS™; Transmedics®) is the only normothermic ex-vivo perfusion system available clinically and which is the most similar to the claimed invention in technical essence and has been chosen as a prototype. It is a portable system allowing normothennic oxygenated blood to perfuse the donor heart, which continues to be beating state outside the body (U.S. Pat. Nos. 9,301,519B2, 7,651,835B2).

This system includes a compact wireless monitor, a special perfusion module, and the necessary solutions. The aorta and pulmonary artery of the donor heart are cannulated and the heart is connected to the OCS device. In the OCS, oxygenated blood is pumped into the aorta, perfusing the coronary arteries. The coronary sinus blood flow then passes through the tricuspid valve (as both the superior and inferior vena cavae are surgically closed) and is ejected by the right ventricle into a pulmonary artery catheter and returns to the blood reservoir. For the primary volume of the perfusion module the donor blood (1200-1500 mL) and special solution is used. The standard method to arrest the donor heart is giving hypothermic (4° C.) crystalloid cardioplegic solution such as Custodiol 1000 mL After connecting the heart to the OCS perfusion system and restoration of normal heart function, the piston-type pump maintains only non-linear perfiisate flow to adjust the mean aortic pressure between 60 mmHg and 90 mmHg and coronary flow between 650 mL/min and 850 mL/min The coronary circulation is supported by isotonic solution with electrolytes, amino acids, insulin and adenosine at a rate of 0-30 mL/hour, epinephrine 2-10 mcg/min injected into perfusate. A special gas mixture with a flow of 250-300 mL/min is used to maintain oxygenation of the heart. Gas and acid-base state of the perfiisate is routinely analyzed during system operation to assess the adequacy of the heart blood supply. It should be noted that if the venous lactate level exceeds 5 mmol/mL the organ is deemed unsuitable for further use. Samples are analyzed by means of a portable analyzer.

### SUMMARY OF THE INVENTION

Several fundamental disadvantages of the above-described prototype system limit its use for prolonged perfusion during conditioning and treatment of the donor heart, namely:
1. The system provides nonlinear blood flow only. The system does not provide linear flow, which is necessary for prolonged perfusion of the arrested (non beating) heart.
2. The system is designed for only the beating organ conditioning, which eliminates perfusion of the arrested heart.
3. The system does not provide integration of additional circuits for extracorporeal hemocorrection therapy, namely:
   Hemodiafiltration for removal of excess fluid and some of the metabolites negatively affecting the organ (primarily lactate levels)
   Hemadsorption for reduction of nonspecific inflammation mediators, some metabolites, and harmful products of cell damage (bilirubin, free hemoglobin, etc.).
4. Epy application of low temperature (4° C.) crystalloid solution for cardiac arrest may lead to prolongation of reperfusion time, increased risk of myocardial damage and other adverse effects of crystalloid (non-blood) cold solutions usage.
5. During the use of the system in operation, it is mandatory to maintain the temperature in the range of 34° C., which excludes the possibility of long-term perfusion at other temperature regimes.
6. The use of the system requires the purchase of the UCS device itself and the corresponding consumables, which makes this technology inaccessible to most countries due to its extreme high cost.
7. The latter provision causes alienity of technology to the majority of specialists (cardiac surgeons, perfusionists, anesthesiologists, intensivists, etc.) and the need for special team training.
8. The system is not universal, and thus cannot be integrated with other devices.

Nowadays, there are other designs of donor heart perfusion/conditioning devices:
Device for Perfusion Conservation and Reconditioning of Donor Heart (RU2754592C1)
Device for perfusion conditioning of donor organ (EA202000057A1, EA038473B1)

There are many unresolved issues in the abovementioned devices:
I. For the device of RU2754592C1:
   1. The bubble oxygenator is used. This type is not applicable in modem clinical practice.
   2. The system is open at the level of the bubble oxygenator and containers, which excludes the possibility of long-term preservation.
   3. Perfusate circulation is performed by two small-section roller pumps, that inevitably leads to damage of perfusate form elements and increases the risk of organ dysfunction during prolonged perfusion.
   4. Thermoregulation is performed in two ways—by Peltier elements and a tubular heat exchanger.
   5. Volumetric perfusion ranges from 40 to 230 mL/min, whereas adequate perfusion of the beating heart requires a capacity of up to 900 mL/min,
   6. The device does not provide mobility to perform organ transportation.
   7. The system is not universal, and cannot be integrated with other devices.
II. For the device of EA202000057A1, EA038473B1:
   1. Perfusate circulation is performed by two small-section roller pumps, that inevitably leads to damage of perfusate limn elements and increases the risk of organ dysfunction during prolonged preservation.
   2. The system is open, which eliminates the possibility of prolonged perfusion.
   3. The system does not provide for integration of additional circuits for hemodiafiltration for removal of excess fluid and some of the harmful metabolites affecting the organ (primarily lactate level).
   4. The device is designed to condition only the beating organ, which precludes arrested heart perfusion
   5. The device is not designed for long-term transportation of the organ.
   6. The device is designed for experimental work in the field of biotechnology.
   7. The system is not universal, and cannot lie integrated with other devices.

Thereby, the purpose of the present invention is to develop a compliant universal portable system for donor heart normothermic preservation and long-term transportation, which would eliminate the disadvantages of similar devices mentioned above.

The aims of the invention are the following: providing of effective perfusion of heart in both beating and non-beating conditions, effective hemocorrection therapy of perfusion solution, flow parameters control by switching linear to non-linear flow and vice versa, and mobility of the system.

To solve the task the system is equipped with a special closed integrated box reservoir to place a donor heart in and universal (applicable in routine perfilsion practice) ports for connection of circuit lines.

The specified task is solved, and technical results are achieved by running the system circulation with centrifugal pump, of which the impeller is suspended in a magnetic field thereby not causing damage to the blood formed elements. The capacity of this pump varies from a few milliliters to several liters. Moreover, the functioning of the impeller provides both linear and non-linear flow with different frequency.

The system simultaneously integrates with several circuits for extracorporeal hemocorrection and hemodiafiltration, which also solves the technical task.

The task is also solved by using a normothermic cardioplegic solution based on donor's blood and crystalloid solution in a 5:1 ratio (potassium chloride 4% (30 mL), magnesium sulfate 25% (1.0 mL), lidocaine 2% (2 mL), sodium hydrocarbonate (13 mL) mannitol 15% (6.5 mL)

The technical task of keeping the required temperature of the donor organ is solved by heat exchange at the oxygenator level as the most effective way.

### BRIEF DESCRIPTION OF THE DRAWING

The patent or application file contains at least one drawing executed in color. Copies of this patent or patent application publication with color drawings will be provided by the Office upon request and payment of the necessary fee.

In order to better understand the subject matter that is disclosed herein and to exemplify how it may be carried out in practice, embodiment will now be described, by way of non-limiting example only, with reference to the accompanying drawings, in which:

FIG. 1 schematically illustrates the configuration and operation of the system of the invention; and

FIGS. 2A and 2B are pictures of an experimental setup of the system of the invention.

### DETAILED DESCRIPTION OF EMBODIMENTS

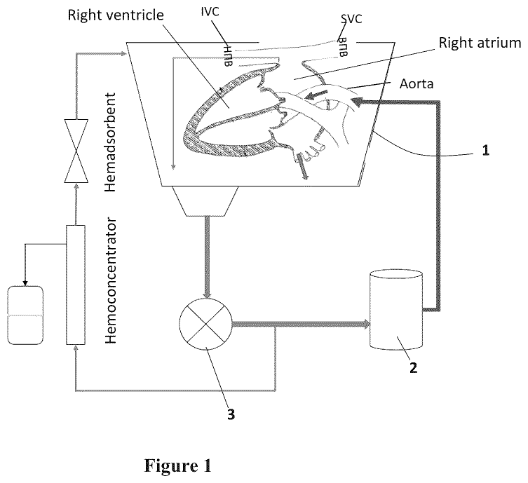

The system of the invention for normothermic ex-situ heart perfusion is schematicafly presented in FIG. 1. The system includes an integrated box reservoir 1 for a donor heart placement, oxygenator 2, centrifugal pump 3 and lines pipes connecting all the components.

It should be noted, although not specifically shown that arrangement/system of connecting pipes may include integrated ports for modules of extracorporeal hemocorrection, hemodiafiltration and laboratory control of perfusate composition.

Thus, according to the invention, in order to place the donor heart in the system, a special closed box with reservoir 1 is used having universal ports for connecting the pipes 4, which are used in routine perfusion practice. The circulation in the system is carried out by one centrifugal pump 3, whose impeller (blade wheel), when functioning, is suspended in a magnetic field and does not cause damage to the formed elements of the blood. The proper temperature of the organ is maintained by implementing heat exchange at the level of the oxygenator 2, as the most effective place.

The performance of the centrifugal pump varies from several milliliters to several liters. Moreover, the functioning of the impeller provides for the provision of laminar flow with different frequencies

It should also be noted that at the same time the system is integrated with several circuits for carrying out extracorporeal hemocorrection and hemodiafiltration.

The method of the ex-situ normothermic heart perfusion functions as follows. The aorta of the heart is cannulated, and connected to the system shown in FIG. 1. The centrifugal pump 3 pumps the oxygenated perfiisate into the aorta under the controlled pressure to maintain the aortic valve closed and coronary arteries perfused. The centrifugal pump flow of the system is adjusted to maintain the mean aortic pressure between 60 mmHg and 80 mmHg and coronary flow between 600 mL/min and 900 mL/min. Venous blood from the heart cavities enters the integrated box reservoir 1, and is pressurized into the oxygenator 2 by the centrifugal pump 3. Then the oxygenated perfiisate is pumped into the aorta, closing the circulatory loop of the system.

The capacity of the device is monitored and controlled by an integrated electronic system. A special gas blender controls oxygen and gas components supply, The system lines provide integrated ports for hemocorrection and hemodiafiltration modules, and for laboratory sampling the perfusate.

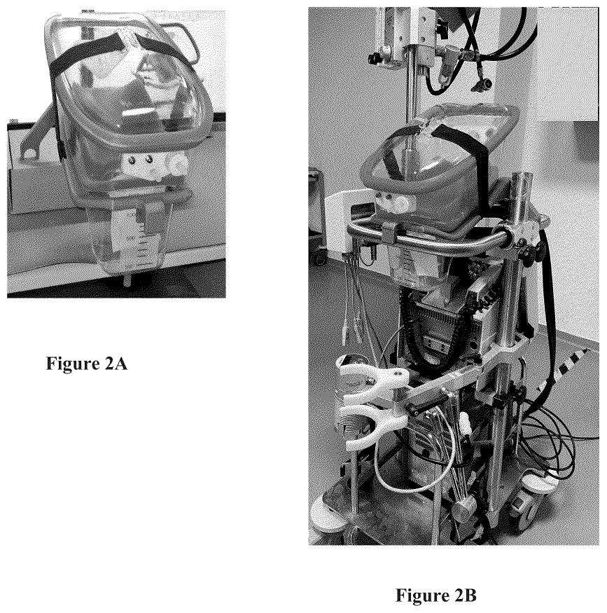

FIGS. 2A and 2B illustrate the experimental setup of the system of the invention. As shown, the system is portable and can be used with conventional equipment, i.e. is a compliant universal system.

1. A system for normothermic preservation and long-term transportation of a donor heart, the system comprising an integrated closed box reservoir for a donor heart placement, an oxygenator, and a centrifugal pump perfbnning perfusion of the heart, said integrated closed box reservoir, oxygenator, and centrifugal pump being interconnected by connecting pipes, enabling venous blood from heart cavities to flow into the integrated box reservoir and being pressurized into the oxygenator by the centrifugal pump, said centrifugal pump haying an impeller configured to be suspended in a magnetic field.

2. The system according to claim 1, wherein said impeller of the centrifugal pump provides linear and non-linear flow with different frequency, thereby enabling control of flow parameters by switching between the linear and non-linear flow.

3. The system according to claim 1, comprising circuits for carrying out extracorporeal hemocorrection and hemodiafiltration,

4. The system according to claim 1, wherein the connecting pipes include integrated ports for connecting one or more modules of extracorporeal hemocorrection, hemodiafiltration and laboratory control of perfusate composition.

5. The system according to claim 1, comprising a normothermic cardioplegic solution based on donor's blood and crystalloid solution in a 5:1 ratio.

6. The system according to claim 5, wherein said solution comprises potassium chloride 4% (30 mL), magnesium sulfate 25% (10 mL), lidocaine 2% (2 mL), sodium hydrocarbonate (131 mL), and mannitol 15% (6.5 mL).

7. The system according to claim 1, configured and operable with heat exchange at the oxygenator level thereby enabling to keep required temperature of the donor heart.

8. The system according to claim 1, configured and operable to provide effective perfusion of the heart in both beating and non-beating conditions, effective hemocorrection therapy of a perfusion solution, and mobility of the system.
